(19) United States
(12) Patent Application Publication
(10) Pub. No.: US 2026/0279493 A1
(43) Pub. Date: Sep. 17, 2026

(54) ARTIFICIAL DIVERSIFICATION APPROACH TO IDENTIFICATION OF CONSERVED SEQUENCES

(71) Applicant: RTX BBN Technologies, Inc., Cambridge, MA (US)

(72) Inventor: Jacob Beal, Iowa City, IA (US)

(21) Appl. No.: 19/569,820

(22) Filed: Mar. 17, 2026

Related U.S. Application Data
(60) Provisional application No. 63/773,046, filed on Mar. 17, 2025.

(51) Int. Cl. G16B 30/00; G01N 33/68; G16B 15/20; G16B 40/00

(52) U.S. Cl. CPC G16B 30/00; G01N 33/6803; G01N 33/6818; G16B 15/20; G16B 40/00

(57) ABSTRACT

Implementations are disclosed that determine identifier sequences of target proteins that correspond to signatures that can be used to determine variants of the target proteins having the same or similar characteristics and/or functionality as the target proteins. The identifier sequences can be determined based on conserved positions of the target protein sequences. The conserved positions can be identified according to a frequency of occurrence of amino acids at individual positions of the variant protein sequences.

### PRIORITY CLAIM

This application claims priority to U.S. provisional patent application Ser. No. 63/773,046, entitled “AN ARTIFICIAL DIVERSIFICATION APPROACH TO IDENTIFICATION OF CONSERVED SEQUENCES” filed Mar. 17, 2025, which is incorporated by reference herein in its entirety.

### INCORPORATION BY REFERENCE OF SEQUENCE LISTING

This application contains a Sequence Listing which has been submitted electronically in ST26 format and hereby incorporated by reference in its entirety. Said ST26 file, created on Mar. 17, 2026, is named 3547071US1.xml and is 8,192 bytes in size

### BACKGROUND

Emerging protein design tools pose a biosecurity threat because they have the potential to create proteins with equivalent or enhanced functions to existing sequences of concern (SoCs) (e.g., controlled toxins and key proteins in controlled pathogens), yet have sequences that are different from those of pre-existing SoCs. Since current biosecurity sequence screening methods primarily evaluate nucleic acid or protein sequences for threat content by comparing them to known pre-existing SoCs, either by relative sequence homology (e.g., ThreatSeq/UltraSeq, Aclid, SeqScreen, NTI common mechanism) or by exact matching of k-mers (e.g., FAST-NA, Secure DNA), this means SoCs may be difficult for such tools to detect.

Protein design tools may be useful not just for looking for SoCs in orders, but also looking for SoCs in physical instantiations with real organisms. Examples include identification of threats in sequencing data. Other non-biosecurity applications are of interest as well, such as identifying diagnostic or therapeutic targets that will remain stable even as a natural disease evolves.

### SUMMARY

Protein design is rapidly improving. Current screening tools use similarity to known sequences of concern: Homology: ThreatSeq/UltraSeq, Aclid, SeqScreen, NTI Common Mechanism; and K-mer: FAST-NA, Secure DNA. Future systems may implement functions of concern with little sequence similarity. Effective techniques having the ability to discover critical amino acids within proteins for other applications, such as enzyme engineering for biomanufacturing, or even just fundamental research and discovery, may be desired. Disclosed are systems and methods to neutralize threats from artificial protein designs. The systems and methods described herein may neutralize the protein design threat by identifying elements that are to remain stable when a protein is designed in order for the protein to have the intended structure. Furthermore, function may be considered as well. For example, SARS-COV-2 spike proteins have to have a specific shape in order to bind to the ACE2 receptor to invade cells. Since the target structure is not being re-engineered, the physics of protein-protein interaction puts strict limits on what protein sequences can possibly have a structure equivalent to that of the SARS-COV-2 spike protein. Furthermore, the existence of conserved sequences suggests there are critical regions where the possible options are tightly constrained. The systems and methods described herein support identifying these regions and, based on the identified regions, building signatures that match broadly across possible sequences for a given target.

### BRIEF DESCRIPTION OF THE DRAWINGS

FIG. 1 illustrates an example framework to analyze variant protein sequences in relation to target protein sequences to determine non-contiguous sequences of the target protein that are present in the variant protein sequences, in accordance with one or more illustrative implementations.

FIG. 2 illustrates an example process to identify positions of a target protein sequence that correspond to a number of positions of variant protein sequences to determine one or more conserved positions, in accordance with one or more illustrative implementations.

FIG. 3 is a flow diagram of an example process to analyze variant protein sequences in relation to target protein sequences to determine non-contiguous sequences of the target protein that are present in the variant protein sequences, in accordance with one or more illustrative implementations.

FIG. 4 illustrates a diagrammatic representation of a machine in the form of a computer system within which a set of instructions may be executed for causing the machine to perform any one or more of the methodologies discussed herein, according to an example implementation.

FIG. 5 is a table showing conserved positions of a target protein sequence within a number of variant protein sequences.

### DETAILED DESCRIPTION

A detailed description of one or more implementations of the disclosed apparatus and method are presented herein by way of exemplification and not limitation with reference to the Figures.

Disclosed are systems and methods to neutralize threats from artificial protein designs. The systems and methods described herein may neutralize the protein design threat by identifying elements that are to remain stable when a protein is designed in order for the protein to have the intended structure. Furthermore, function may be considered as well. For example, SARS-COV-2 spike proteins have to have a specific shape in order to bind to the ACE2 receptor to invade cells. Since the target structure is not being re-engineered, the physics of protein-protein interaction puts limits on what protein sequences can possibly have a structure equivalent to that of the SARS-CoV-2 spike protein. Furthermore, the existence of conserved sequences suggests there are regions where the possible options are tightly constrained. The systems and methods described herein support identifying these regions and, based on the identified regions, building signatures that match broadly across possible sequences for a given target.

In implementations, disclosed is an artificial diversification approach to the identification of conserved sequences workflow for generating signatures to detect variants with selected properties similar to those of the target protein. Specifically, aspects of the artificial diversification approach for identification of conserved sequences as described herein may first use protein design tools to generate a highly diverse library of candidate alternative designs. The artificial diversification approaches described herein can implement machine learning techniques to generate large numbers of variant sequences for a given target sequence. Naturally-occurring protein sequences do not provide sufficient diversity to generate signature sequences that can be used to identify variants of target sequences having a same or similar functionality or characteristics. Thus, the large number of sequences and the high level of variation of the artificially generated sequences results in the ability to identify conserved positions of the target protein.

Optionally, in some implementations, some candidate alternative designs may be eliminated from consideration by the use of tools such as, for example, folding models, binding models, or in vitro assays, to determine designs that cannot have the selected properties of the target protein. Next, analysis of the set of candidate alternative designs (e.g., bioinformatic sequence analysis) identifies conserved amino acids that are strongly associated with the target properties. Finally, systems may generate “signature” patterns that provide sensitive and specific detection of the target properties in novel sequences. In some non-limiting examples, the systems and techniques described herein may incorporate AI-based techniques with respect to the protein design tools and generating signatures (e.g., AI-resistant signatures), but implementations of the present disclosure are not limited thereto.

In implementations, physical constraints may be identified that are restrictive and specific enough for use in biosecurity assessments. Implementations may provide exploration of this space that is computationally feasible through non-AI-assisted or AI-assisted implementations. This can allow for a qualitative transformation of biosecurity, biosurveillance, and biodefense from a “reactive” race against emerging threats toward a “proactive” posture where defenses are aimed against all possible threats with a given set of properties.

Furthermore, implementations may upgrade signatures by applying constraint-solving and theorem-proving tools to prove that the signatures cover all physically realizable FoC sequences.

Finally, in addition to synthesis screening, the signatures generated in accordance with the techniques described herein (e.g., AI-resistant and AI-proof signatures, but not limited thereto) may also be applicable to other related sequence interpretation challenges, such as air and water monitoring and healthcare diagnostics. Depending on the specifics of the conserved amino acids, it can be possible to use signatures generated in accordance with the techniques described herein in the development of broad-spectrum vaccines, diagnostics, and therapeutics that cannot be evaded without the threat losing its function of concern.

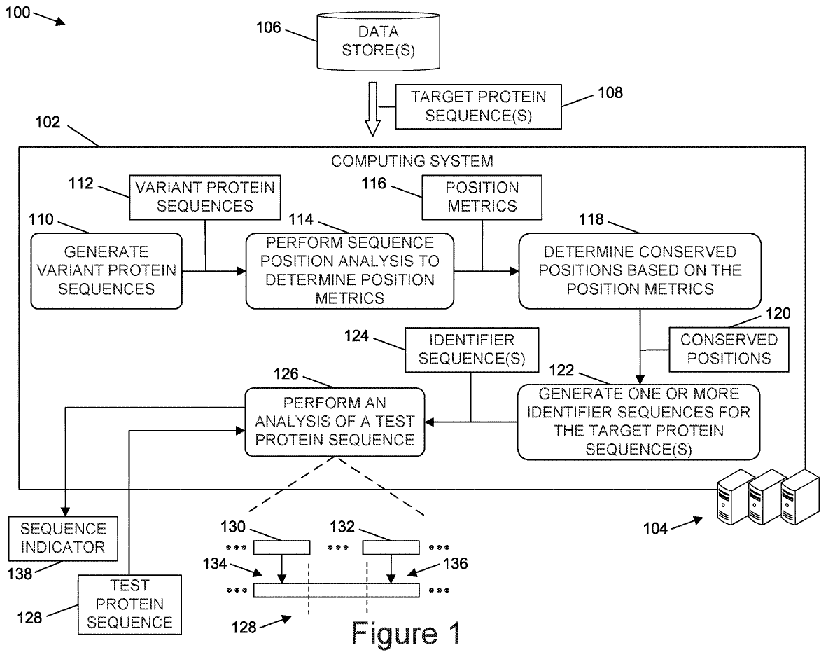

FIG. 1 illustrates an example framework 100 to analyze variant protein sequences in relation to target protein sequences to determine non-contiguous sequences of the target protein that are present in the variant protein sequences, in accordance with one or more illustrative implementations. In various examples, FIG. 1 illustrates aspects of an artificial diversification approach to identification of conserved sequences workflow for generating signatures to detect whether a given protein is likely to have similar properties to those of a target threat protein, in accordance with one or more implementations of the present disclosure. In a non-limiting example, the given protein may be a spike protein in isolation. The workflow at FIG. 1 may include threat protein selection, design diversification (i.e., determining or generating a diversified collection of sequences), conserved amino acid identification (e.g., identifying conserved amino acids), signature generation based on the conserved amino acids, and biosecurity (e.g., threat detection) based on the generated signatures. In some implementations, design diversification may be implemented using AI-based techniques or other techniques supportive of design diversification. In some aspects, the conserved amino acids may correspond to toxins which are related to the threat protein identified at the start of the workflow.

FIG. 1 also illustrates aspects of an artificial diversification approach to identification of conserved sequences workflow for generating signatures that includes removal of some designs from consideration, in accordance with one or more implementations of the present disclosure. In this case, the properties of interest include the protein's ability to bind to a host protein, and designs are removed from consideration if a protein folding model indicates that the design is unlikely to fold to a similar structure to the target protein or if protein binding models indicate that the design is unlikely to bind to the host protein. In one or more illustrative examples, the given protein can be a spike protein in combination with a human receptor. In some aspects, the approach may replace or combine modeling with other forms of assessment, such as, for example, empirical in vitro assays.

In accordance with one or more implementations of the present disclosure, a method is provided for defending against re-engineered toxins, using an artificial diversification approach to identification of conserved sequences.

The method includes using protein design methods to generate highly divergent likely-functional variants. In some aspects, the protein design methods may be AI-based, but are not limited thereto.

The method includes identifying conserved non-contiguous patterns in the generated variants.

The method includes adapting non-contiguous signature matching methods for synthesis screening.

The method includes integrating into FAST-NA-scanner deployments.

The method includes disseminating advisories to other screening organizations via IGSC and test set/SoC effort.

The framework 100 can include a computing system 102 that analyzes one or more target protein sequences and a number of variant protein sequences to determine protein sequences that can be used to identify at least one variant of the target protein sequence. The computing system 102 can be implemented by one or more computing devices 104. The one or more computing devices 104 can include one or more server computing devices, one or more desktop computing devices, one or more laptop computing devices, one or more tablet computing devices, one or more mobile computing devices, or combinations thereof. In certain implementations, at least a portion of the one or more computing devices 104 can be implemented in a distributed computing environment. For example, at least a portion of the one or more computing devices 104 can be implemented in one or more cloud computing architectures.

The framework 100 can also include one or more data stores 106. The one or more data stores 106 can include one or more data stores that are electronically accessible by the computing system 102. For example, the computing system 102 can access the one or more data stores 106 using at least one of one or more wireless communication networks or one or more wired communication networks. In one or more implementations, at least a portion of the one or more data stores 106 can be remotely located with respect to the computing system 102. Additionally, at least a portion of the one or more data stores 106 can be maintained by a same entity that implements the computing system 102. Further, at least a portion of the one or more data stores 106 can be maintained by a different entity than the entity that maintains the computing system 102. The one or more data stores 106 can include one or more publicly accessible databases. The one or more data stores 106 can also include one or more privately maintained databases. In various implementations, the data stored by the one or more data stores 106 can be accessed using one or more websites.

One or more target protein sequences 108 can be obtained from the one or more data stores 106. In one or more additional examples, at least a portion of the one or more target protein sequences 108 can be obtained by user input via one or more user interfaces. In one or more examples, the one or more target protein sequences 108 can correspond to one or more viral proteins. In one or more additional examples, the one or more target protein sequences 108 can correspond to one or more bacterial proteins. In one or more further examples, the one or more target protein sequences 108 can correspond to one or more fungal proteins. In still other examples, the one or more target protein sequences 108 can correspond to one or more toxins. In various examples, the one or more target protein sequences 108 can include proteins that correspond to naturally occurring proteins. Additionally, the one or more target protein sequences 108 can correspond to one or more synthetically produced proteins.

In one or more illustrative examples, the one or more target protein sequences 108 can include alphanumeric representations of amino acids included in one or more proteins. The alphanumeric representations of proteins can include, for individual positions of a protein, one or more alphanumeric representations of the amino acid located at the individual positions. In at least some examples, the alphanumeric representations of amino acids located at individual positions of the one or more target protein sequences 108 can include a single letter or symbol. In one or more additional examples, the alphanumeric representations of amino acids located at individual positions of the one or more target protein sequences 108 can include multiple letters or symbols. To illustrate, an “A” or “Ala” can be used to represent alanine at positions of the one or more target protein sequences 108, and a “K” or “Lys” can be used to represent lysine at positions of the one or more target protein sequences 108.

The computing system 102 can, at operation 110, generate variant protein sequences 112. The variant protein sequences 112 can include changes at one or more positions of the one or more target protein sequences 108. In one or more examples, the variant protein sequences 112 can include changes for at least 5%, at least 10%, at least 20%, at least 30%, at least 40%, at least 50%, at least 60%, or at least 70% of the positions of the one or more target protein sequences 108. In one or more illustrative examples, the variant protein sequences 112 can have from about 30% to about 95%, from about 30% to about 80%, from about 30% to about 70%, from about 30% to about 60%, from about 30% to about 50%, from about 30% to about 40%, from about 40% to about 95%, from about 40% to about 80%, from about 40% to about 70%, from about 40% to about 60%, from about 50% to about 95%, from about 50% to about 80%, from about 50% to about 70%, from about 50% to about 60%, from about 60% to about 95%, from about 60% to about 80%, from about 60% to about 70%, from about 70% to about 95%, or from about 80% to about 95% sequence identity with respect to the one or more target protein sequences 108. In at least some examples, the computing system 102 can generate from 100 to 5,000,000 variant amino acid sequences; from 1000 to 1,000,000 variant amino acid sequences; from 10,000 to 500,000 variant amino acid sequences; from 10,000 to 250,000 variant amino acid sequences; from 10,000 to 100,000 variant amino acid sequences; from 50,000 to 500,000 variant amino acid sequences; from 50,000 to 250,000 variant amino acid sequences; from 50,000 to 100,000 variant amino acid sequences; from 100,000 to 500,000 variant amino acid sequences; or from 100,000 to 250,000 variant amino acid sequences.

In various examples, the variant protein sequences 112 can correspond to variant proteins that maintain at least a portion of the secondary structures of one or more target proteins that correspond to the one or more target protein sequences 108. Additionally, the variant protein sequences 112 can correspond to variant proteins that maintain at least a portion of the tertiary structures of one or more target proteins that correspond to the one or more target protein sequences 108. The variant protein sequences 112 can also correspond to variant proteins that maintain at least a portion of a three-dimensional folding configuration of the one or more target proteins that correspond to the one or more target protein sequences 108.

In one or more examples, the variant protein sequences 112 can correspond to variant proteins that maintain at least a portion of the biophysical properties of the one or more target proteins that correspond to the one or more target protein sequences 108. To illustrate, the variant protein sequences 112 can correspond to variant proteins that maintain solubility characteristics of the one or more target proteins that correspond to the one or more target protein sequences 108. In one or more additional examples, the variant protein sequences 112 can correspond to variant proteins that maintain thermostability properties of the one or more target proteins that correspond to the one or more target protein sequences 108. In still other examples, the variant protein sequences 112 can correspond to variant proteins that maintain molecular weights of the one or more target proteins that correspond to the one or more target protein sequences 108. Variant proteins corresponding to the variant protein sequences 112 can maintain a biophysical property of one or more target proteins of the one or more target protein sequences 108 based on values of biophysical properties of the variant proteins corresponding to the variant protein sequences 112 being within from about 1% to about 20%, from about 1% to about 15%, from about 1% to about 10%, from about 1% to about 5%, from about 5% to about 20%, from about 5% to about 15%, or from about 5% to about 10% of the values of the biophysical properties of the target proteins corresponding to the one or more target protein sequences 108.

Further, the variant protein sequences 112 can correspond to variant proteins that maintain at least a portion of the functionality of one or more target proteins that correspond to the one or more target protein sequences 108. In one or more examples, variant proteins corresponding to the one or more variant protein sequences 112 can maintain at least a portion of the functionality of target proteins corresponding to the one or more target protein sequences 108 based on maintaining at least a portion of the secondary structures, tertiary structures, folding configurations, and/or biophysical properties of target proteins that correspond to the one or more target protein sequences 108. For example, in situations where a target protein corresponding to the one or more target protein sequences 108 has one or more active sites, variant proteins corresponding to the variant protein sequences 112 can include at least a portion of the one or more active sites of the target protein. In other examples, in situations where a target protein corresponding to the one or more target protein sequences 108 has one or more ligand-binding sites, variant proteins corresponding to the variant protein sequences 112 can include at least a portion of the one or more ligand-binding sites of the target protein. In one or more illustrative examples, in situations where a target protein corresponding to the one or more target protein sequences 108 includes an antibody having one or more antigen-binding sites, variant proteins corresponding to the variant protein sequences 112 can include at least a portion of the one or more antigen-binding sites of the target protein.

In various examples, the variant protein sequences 112 can be generated by executing one or more machine learning models with respect to the one or more target protein sequences 108. For example, the computing system 102 can execute one or more machine learning models to generate the variant protein sequences 112 by changing amino acids located at a number of positions of the one or more target protein sequences 108. In one or more examples, the computing system 102 can execute one or more transformer-based machine learning models to generate the variant protein sequences 112 based on the one or more target protein sequences 108. In one or more additional examples, the computing system 102 can execute one or more graph-based neural networks to generate the variant protein sequences 112 based on the one or more target protein sequences 108. In one or more further examples, the computing system 102 can execute one or more variational autoencoder machine learning models to generate the variant protein sequences 112 based on the one or more target protein sequences 108. In still other examples, the computing system 102 can execute one or more generative adversarial networks to generate the variant protein sequences 112 based on the one or more target protein sequences 108.

In one or more illustrative examples, the computing system 102 can execute the one or more machine learning models with respect to one or more parameters and/or one or more criteria to generate the variant protein sequences 112 based on the one or more target protein sequences 108. For example, the computing system 102 can execute one or more machine learning models based on one or more parameters relating to biophysical properties to generate variant protein sequences 112 corresponding to variant proteins having biophysical properties that are the same or similar to biophysical properties of one or more target proteins corresponding to the one or more target protein sequences 108. The computing system 102 can also execute one or more machine learning models based on one or more parameters relating to one or more secondary structures to generate variant protein sequences 112 corresponding to variant proteins having tertiary structures that are the same or similar to tertiary structures of one or more target proteins corresponding to the one or more target protein sequences 108. Additionally, the computing system 102 can also execute one or more machine learning models based on one or more parameters relating to one or more tertiary structures to generate variant protein sequences 112 corresponding to variant proteins having secondary structures that are the same or similar to secondary structures of one or more target proteins corresponding to the one or more target protein sequences 108. In still other examples, the computing system 102 can execute one or more machine learning models based on one or more parameters relating to protein folding to generate variant protein sequences 112 corresponding to variant proteins having secondary structures that are the same or similar to protein folding of one or more target proteins corresponding to the one or more target protein sequences 108. Further, the computing system 102 can execute one or more machine learning models based on one or more parameters relating to protein functionality to generate variant protein sequences 112 corresponding to variant proteins having functionality that is the same or similar to functionality of one or more target proteins corresponding to the one or more target protein sequences 108.

The one or more parameters used by the one or more machine learning models executed by the computing system 102 to generate the variant protein sequences 112 can indicate that at least a threshold number of positions of the one or more target protein sequences 108 are modified to generate the variant protein sequences 112. The one or more parameters used by the one or more machine learning models executed by the computing system 102 to generate the variant protein sequences 112 can indicate that at least a threshold number of positions of the one or more target protein sequences 108 are conserved. In one or more illustrative examples, the one or more parameters used by the one or more machine learning models executed by the computing system 102 to generate the variant protein sequences 112 can indicate that from 5% to 70%, from 5% to 60%, from 5% to 50%, from 5% to 40%, from 5% to 30%, from 5% to 20%, from 10% to 70%, from 10% to 60%, from 10% to 50%, from 10% to 40%, from 10% to 30%, from 10% to 20%, from 20% to 70%, from 20% to 60%, from 20% to 50%, from 20% to 40%, from 20% to 30%, from 30% to 70%, from 30% to 60%, from 30% to 50%, from 30% to 40%, from 40% to 70%, from 40% to 60%, from 40% to 50%, from 50% to 70%, from 50% to 60%, or from 60% to 70% of the positions of the one or more target protein sequences 108 are modified to produce the variant protein sequences 112.

In various examples, the one or more parameters used by the one or more machine learning models to generate the variant protein sequences 112 can indicate that the variant protein sequences 112 include a same or similar number of amino acids as the one or more target protein sequences 108. To illustrate, the one or more parameters used by the one or more machine learning models executed by the computing system 102 to generate the variant protein sequences 112 can indicate that a number of amino acids included in the variant protein sequences 112 is within from about 0.5% to about 10%, from about 0.5% to about 8%, from about 0.5% to about 5%, from about 0.5% to about 3%, from about 1% to about 10%, from about 1% to about 8%, from about 1% to about 5%, from about 1% to about 3%, from about 3% to about 10%, from about 3% to about 8%, from about 3% to about 5%, from about 5% to about 10%, from about 5% to about 8%, or from about 8% to about 10% of the number of amino acids included in the one or more target protein sequences 108. In one or more illustrative examples, the variant protein sequences 112 generated by the computing system 102 executing one or more machine learning models can include from 30 amino acids to 40,000 amino acids, from 50 amino acids to 10,000 amino acids, from 100 amino acids to 1000 amino acids, from 200 amino acids to 800 amino acids, or from 300 amino acids to 600 amino acids.

In at least some examples, the computing system 102 can execute one or more first machine learning models to generate a number of variant protein sequences and the computing system 102 can execute one or more second machine learning models to determine a subset of the variant protein sequences 112. In this way, a pipeline of machine learning models can be implemented by the computing system 102 to determine the variant protein sequences 112. In one or more examples, the one or more first machine learning models can include at least one of one or more graph-based neural network machine learning models, one or more transformer-based machine learning models, one or more variational autoencoders, or one or more generative adversarial networks. Additionally, the one or more second machine learning models can include at least one of one or more diffusion-based machine learning models, one or more transformer-based machine learning models, or one or more graph-based neural networks. In various examples, the one or more first machine learning models can be different from the one or more second machine learning models. Additionally, the one or more first machine learning models can be trained in a different manner from the one or more second machine learning models. To illustrate, the one or more first machine learning models can be trained on thousands up to millions of protein sequences to generate a number of variant protein sequences based on at least one target protein sequence. The one or more second machine learning models can be trained on thousands up to millions of protein sequences as well as folding information, configuration information, and/or three-dimensional structural information to determine protein sequences having specified functionality, three-dimensional structures, secondary structures, folding configurations, and/or biophysical properties. In one or more illustrative examples, the computing system 102 can execute one or more protein language models to determine the variant protein sequences 112.

In still other examples, the one or more first machine learning models can generate a number of variant protein sequences and variant proteins can be synthesized that correspond to at least a portion of the number of variant protein sequences generated by the one or more first machine learning models. In these scenarios, the synthesized variant proteins can be subjected to one or more assays and/or one or more analytical tests. To illustrate, the synthesized variant proteins can be subjected to one or more assays to determine binding between the synthesized variant proteins and a ligand that binds to target proteins corresponding to the one or more target protein sequences 108. In various examples, the binding characteristics and/or the biophysical properties of the synthesized variant proteins can be used to determine a subset of the number of variant protein sequences generated by the one or more machine learning models to determine the variant protein sequences 112. For example, the variant protein sequences 112 can include amino acid sequences of synthesized variant proteins that have at least a threshold amount of binding with a ligand that binds to one or more target proteins. Further, the variant protein sequences 112 can include amino acid sequences of synthesized variant proteins having the same or similar biophysical properties as one or more target proteins based on results of analytical tests performed with respect to the synthesized variant proteins.

At operation 114, the computing system 102 can perform a sequence position analysis to determine position metrics 116. In one or more examples, the computing system 102 can analyze amino acids present at individual positions of the one or more target protein sequences 108 and the variant protein sequences 112 to determine the position metrics 116. In at least some examples, the computing system 102 can perform one or more alignment operations between the one or more target protein sequences 108 and the variant protein sequences 112 to determine the position metrics 116. In various examples, the computing system 102 can analyze the amino acids present at individual positions of the one or more target protein sequences 108 with respect to homologous positions of the variant protein sequences 112 to determine a frequency of occurrence of a given amino acid at the individual positions. In one or more illustrative examples, the computing system 102 can determine the position metrics 116 by determining at least one of a frequency or a probability of a given amino acid being present at an individual position of the variant protein sequences 112 with respect to the amino acid present at the homologous position of the one or more target protein sequences 108. For example, the computing system 102 can determine position metrics 116 for individual positions of a target protein sequence 108, such as a fifth position of a target protein sequence 108, by determining the amino acid present at the homologous position, such as the fifth position, of the variant protein sequences 112. In at least some examples, the position metric 116 can indicate a frequency of occurrence for each of the standard amino acids across individual positions of the variant protein sequences 112 in relation to the homologous positions of the one or more target protein sequences 108. In one or more additional illustrative examples, the computing system 102 can, at operation 114, analyze tens up to hundreds or thousands of individual positions for thousands up to millions of variant protein sequences 112 to determine the position metrics 116.

The computing system 102 can, at operation 118, determine conserved positions 120 of the one or more target sequences 108 based on the position metrics 116. In one or more examples, the conserved positions 120 can include positions of the one or more target protein sequences 108 having a position metric that satisfies one or more criteria. For example, the computing system 102 can determine that a specified amino acid is present at an individual position of a target protein sequence 108. The computing system 102 can also determine that a frequency of occurrence of the specified amino acid at homologous positions of the variant protein sequences 112 is at least a threshold frequency. In these scenarios, the computing system 102 can determine that the individual position of the target protein sequence 108 is a conserved position 120. In various examples, the conserved positions 120 can comprise one or more sets of non-contiguous amino acids of the one or more target protein sequences 108. Sets of non-contiguous amino acids can be separated by one or more amino acids that are located in positions of the one or more target amino acid sequences that are between conserved positions 120 located sequentially along the one or more target protein sequences 108.

In one or more illustrative examples, the computing system 102 can determine that a position of a target protein sequence 108 is a conserved position 120 in response to determining that a frequency of occurrence of the amino acid at homologous positions of the variant protein sequences 112 is from about 20% to about 90%, from about 20% to about 70%, from about 20% to about 50%, from about 40% to about 90%, from about 40% to about 70%, from about 50% to about 90%, from about 50% to about 70%, from about 60% to about 90%, or from about 70% to about 90%. In one or more additional illustrative examples, from about 1% to about 80%, from about 1% to about 60%, from about 1% to about 40%, from about 1% to about 20%, from about 5% to about 80%, from about 5% to about 60%, from about 5% to about 40%, from about 5% to about 20%, from about 10% to about 80%, from about 10% to about 60%, from about 10% to about 40%, from about 10% to about 20%, from about 20% to about 80%, from about 20% to about 60%, from about 20% to about 40%, from about 40% to about 80%, from about 40% to about 60%, or from about 60% to about 80% of the positions of the one or more target protein sequences 108 are included in the conserved positions 120. In one or more additional illustrative examples, the conserved positions 120 can comprise from 2 to 500 amino acids, from 2 to 250 amino acids, from 2 to 100 amino acids, from 2 to 50 amino acids, from 2 to 25 amino acids, from 2 to 10 amino acids, from 10 to 500 amino acids, from 10 to 250 amino acids, from 10 to 100 amino acids, from 10 to 50 amino acids, from 10 to 25 amino acids, from 25 to 500 amino acids, from 25 to 250 amino acids, from 25 to 100 amino acids, from 25 to 50 amino acids, from 50 to 500 amino acids, from 50 to 250 amino acids, from 50 to 100 amino acids, from 100 to 500 amino acids, from 100 to 250 amino acids, and from 250 to 500 amino acids.

At operation 122, the computing system 102 can generate one or more identifier sequences 124 for the target protein sequences 108 based on the conserved positions 120. In at least some examples, the one or more identifier sequences 124 can be used to identify sequences of proteins having at least one of a same or similar functionality, three-dimensional conformation, folding configuration, secondary structures, or biophysical properties of the one or more target sequences 108. In one or more examples, the one or more identifier sequences 124 can be comprised of the conserved positions 120. In one or more illustrative examples, the one or more identifier sequences 124 can include a single amino acid sequence comprised of each of the conserved positions 120. In one or more additional illustrative examples, the one or more identifier sequences 124 can include multiple amino acid sequences with individual amino acid sequences corresponding to the identifier sequences 124 being comprised of a group of the conserved positions 120. In situations where the conserved positions 120 are included in multiple identifier sequences 124, individual identifier sequences 124 can include from 1 to 100 amino acids, from 1 to 50 amino acids, from 1 to 25 amino acids, from 1 to 10 amino acids, from 1 to 5 amino acids, from 5 to 100 amino acids, from 5 to 50 amino acids, from 5 to 25 amino acids, from 5 to 10 amino acids, from 10 to 100 amino acids, from 10 to 50 amino acids, from 10 to 25 amino acids, from 25 to 100 amino acids, from 25 to 50 amino acids, or from 50 to 100 amino acids. In various examples, the one or more identifier sequences 124 can include at least one non-contiguous section of an individual target protein sequence 108 that is comprised of at least a portion of the conserved positions 120. In one or more further illustrative examples, the one or more identifier sequences 124 can be arranged according to the order in which the individual identifier sequences 124 are arranged along individual target protein sequences 108 from the N-terminus of an individual target protein sequence 108 to a C-terminus of the individual target protein sequence 108.

The computing system 102 can, at operation 126, perform an analysis of a test protein sequence 128 based on the one or more identifier sequences 124. In one or more examples, the computing system 102 can obtain the test protein sequence 128 from at least one of one or more additional data stores, one or more portable memory devices, one or more computing devices, or one or more user interfaces. In addition, the test protein sequence 128 can be obtained from one or more sequencing devices. For example, a sample can be obtained from an environment and proteins can be extracted from the sample. In these situations, the proteins can be supplied to a sequencing device and the sequencing device can output the test protein sequence 128 that is subsequently provided to the computing system 102. In various examples, the test protein sequence 128 can be directly provided to the computing system 102 via at least one of wired or wireless electronic communication. In one or more illustrative examples, the test protein sequence 128 can be generated by a nanopore sequencing device, a fluorosequencing device, an Edman degradation sequencing device, or a mass spectrometer. In at least some examples, the test protein sequence 128 can be provided to the computing system 102 in real-time from a sequencing device.

The analysis of the test protein sequence 128 based on the one or more identifier sequences 124 can be performed to determine whether or not the test protein sequence 128 corresponds to at least one target protein sequence 108 with respect to at least one of functionality, secondary structures, tertiary structures, folding configuration, or biophysical properties. In one or more illustrative examples, the computing system 102 can analyze amino acids present at individual positions of the test protein sequence 128 with respect to the one or more identifier sequences 124. In at least some examples, in addition to determining whether individual identifier sequences 124 are present in the test protein sequence 128, the computing system 102 can also determine whether or not the order of the amino acids of the test protein sequence 128 corresponds to an order of the amino acids of the one or more identifier sequences 124.

In one or more illustrative examples, the one or more identifier sequences 124, such as a first example identifier sequence 130 and a second example identifier sequence 132, can be arranged from the N-terminus to a C-terminus of a target protein sequence 108, and the test protein sequence 128 can be arranged from the N-terminus to the C-terminus. In various examples, the computing system 102 can analyze the amino acids included in positions of the test protein sequence 128 with respect to amino acids of the one or more identifier sequences 124 to determine matches between the one or more identifier sequences 124 and one or more portions of the test protein sequence 128. In the illustrative example of FIG. 1, the computing system 102 can determine that a first segment 134 of the test protein sequence 128 corresponds to the first example identifier sequence 130 and that a second segment 136 of the test protein sequence 128 corresponds to the second example identifier sequence 132 and that the first segment 134 and the second segment 136 of the test protein sequence 128 are arranged in an order that corresponds to an order in which the first example identifier sequence 130 and the second example identifier sequence 132 are arranged along a target protein sequence 108. In at least some examples, a gap between the first segment 134 and the second segment 136 of the test protein sequence 128 can include a same number of amino acids as a gap between the first example identifier sequence 130 and the second example identifier sequence 132 along a target protein sequence 108. In one or more additional examples, a gap between the first segment 134 and the second segment 136 of the test protein sequence 128 can include a different number of amino acids than a gap between the first example identifier sequence 130 and the second example identifier sequence 132 along a target protein sequence 108.

In one or more examples, the computing system 102 can implement one or more probabilistic data structures to perform the analysis of the test protein sequence 128 with respect to the one or more identifier sequences 124. In one or more illustrative examples, the computing system 102 can implement a Bloom filter to perform the analysis of the test protein sequence 128 with respect to the one or more identifier sequences 124. In one or more additional illustrative examples, the computing system 102 can implement one or more machine learning techniques to perform the analysis of the test protein sequence 128 with respect to the one or more identifier sequences 124. In various examples, the computing system 102 can determine a number of segments of the test protein sequence 128 that correspond to the one or more identifier sequences 124.

The computing system 102 can generate a sequence indicator 138 based on the analysis of the test protein sequence 128 with respect to the one or more identifier sequences 124. In one or more examples, the sequence indicator 138 can indicate a probability that the test protein sequence 128 corresponds to a protein having the same or similar characteristics as a target protein corresponding to a target protein sequence 108. For example, the computing system 102 can determine the sequence indicator 138 by determining a probability of a protein corresponding to the test protein sequence 128 having the same or similar secondary structures, the same or similar tertiary structures, the same or similar folding configuration, the same or similar functionality, and/or the same or similar biophysical properties as a target protein corresponding to a target protein sequence 108. In one or more illustrative examples, a probability of a protein corresponding to the test protein sequence 128 having the same or similar characteristics as a target protein corresponding to a target protein sequence 108 can be based on an amount of segments of the test protein sequence 128 that correspond to the one or more identifier sequences 124. In at least some examples, as the amount of segments of the test protein sequence 128 that correspond to the one or more identifier sequences 124 increases, the probability of a protein corresponding to the test protein sequence 128 having the same or similar characteristics as a target protein corresponding to a target protein sequence 108 can increase. In one or more additional illustrative examples, the computing system 102 can determine the sequence indicator 138 by determining whether each of the one or more identifier sequences 124 corresponds to a segment of the test protein sequence 128.

In one or more examples, the computing system 102 can perform a training process to determine frequency thresholds for determining conserved positions 120 based on the position metrics 116. For example, the computing system 102 can perform an iteration of the training process by determining a first frequency threshold for positions of the one or more target protein sequences 108 and determining a first number of conserved positions by analyzing the position metrics 116 for the one or more target protein sequences 108 based on the first frequency threshold. In addition, the computing system 102 can determine a first number of identifier sequences based on the first conserved positions and analyze a number of training protein sequences with respect to the first number of identifier sequences. In at least some examples, the number of training protein sequences can correspond to at least one of one or more proteins having the same or similar characteristics as one or more target proteins corresponding to the one or more target protein sequences 108 or one or more additional proteins having different characteristics with respect to characteristics of the one or more target proteins.

The computing system 102 can determine one or more training metrics based on a number of matches of the first number of identifier sequences with the number of training protein sequences, a number of false positives determined by the computing system 102 with respect to training protein sequences that correspond to the one or more target protein sequences 108, probabilities of the training protein sequences corresponding to the one or more target protein sequences 108, or a combination thereof. In situations where the training metrics do not satisfy one or more threshold criteria, the computing system 102 can perform an additional iteration of the training process based on a second frequency threshold for positions of the one or more target protein sequences 108. Iterations of the training process can continue until the training metrics satisfy the one or more threshold criteria. In various examples, the one or more threshold criteria for the training metrics can correspond to a threshold number of matches between the one or more training identifier sequences and segments of the training protein sequences, a maximum number of false positives between the training protein sequences and the one or more target protein sequences 108, at least a threshold number of training proteins corresponding to the training protein sequences having at least a threshold probability of having the same or similar characteristics as target proteins corresponding to the one or more target protein sequences 108, or a combination thereof. In at least some examples, the frequency threshold determined by the training process performed by the computing system 102 can apply to each position of the one or more target protein sequences 108. In one or more additional examples, the frequency threshold determined by the training process can vary for individual positions of the one or more target protein sequences 108.

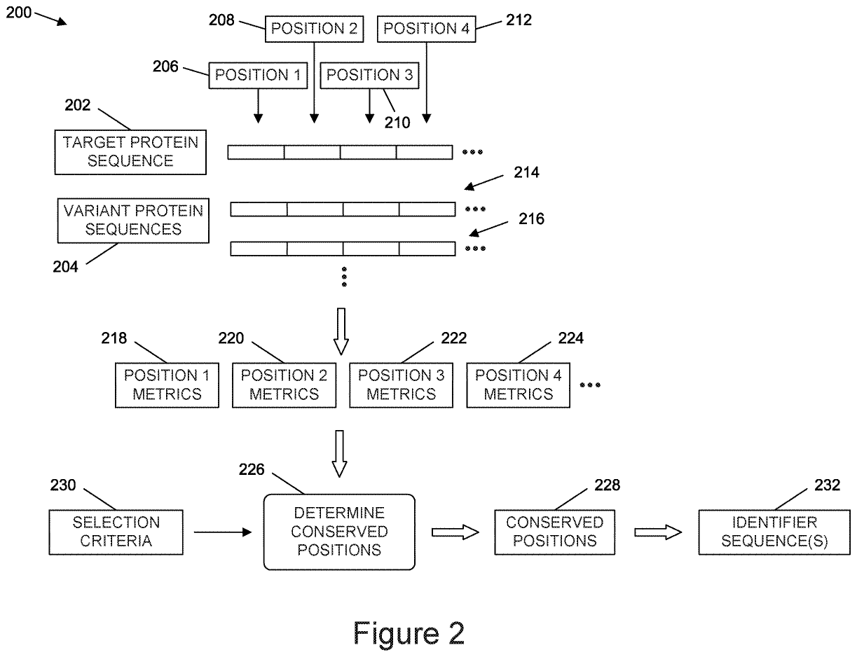

FIG. 2 illustrates an example process 200 to identify positions of a target protein sequence 202 that correspond to a number of positions of variant protein sequences 204 to determine one or more conserved positions, in accordance with one or more illustrative implementations. The illustrative example of FIG. 2 indicates a position one 206, a position two 208, a position three 210, and a position four 212. Although the illustrative example of FIG. 2 shows four amino acid positions, in other examples, the target protein sequence 202 and the variant protein sequences 204 can include a greater number of amino acids. The positions 206, 208, 210, 212 can include amino acid positions of the target protein sequence 202 and corresponding amino acid positions of individual variant protein sequences, such as corresponding amino acid positions of a first example variant protein sequence 214 and a second example variant protein sequence 216. In one or more examples, the amino acid positions of the variant protein sequences 204 that correspond to amino acid positions of the target protein sequence 202 can comprise homologous positions. That is, the position one 206 can indicate the same amino acid position in the target protein sequence 202 and in the variant protein sequences 204. In various examples, the target protein sequence 202 and the variant protein sequences 204 can be arranged from the N-terminus to the C-terminus.

The process 200 can include determining position metrics for individual positions 206, 208, 210, 212. For example, the process 200 can include determining position one metrics 218 that correspond to amino acids present at position one 206. To illustrate, a frequency of individual amino acids at position one 206 can be determined based on the amino acids present at position one 206 of the variant protein sequences 204. In one or more illustrative examples, for each of the twenty naturally occurring amino acids, a number of times that the individual amino acid is present at position one 206 in the variant protein sequences 204 can be used to determine the position one metrics 218. Additionally, a number of times that each of the twenty naturally occurring amino acids is present at position two 208 of the variant protein sequences 204 can be used to determine the position two metrics 220, a number of times that each of the twenty naturally occurring amino acids is present at position three 210 of the variant protein sequences 204 can be used to determine the position three metrics 222, and a number of times that each of the twenty naturally occurring amino acids is present at position four 212 of the variant protein sequences 204 can be used to determine the position four metrics 224. In one or more additional illustrative examples, the position metrics 218, 220, 222, 224 can indicate a percentage of occurrence of individual amino acids at a respective position based on the frequency of occurrence of the individual amino acids at the respective position in the variant protein sequences 204. To illustrate, the position one metrics 218 can indicate that taurine is present at position one 206 in 85% of the variant protein sequences 204, glycine is present at position one 206 in 10% of the variant protein sequences 204, and alanine is present at position one 206 in 5% of the variant protein sequences 204.

At operation 226, the process 200 can include determining conserved positions 228 based on the position metrics 218, 220, 222, 224. The conserved positions 228 can be determined based on the position metrics 218, 220, 222, 224 in conjunction with one or more selection criteria 230. The selection criteria 230 can indicate one or more threshold values for the position metrics in order for a position to be identified as a conserved position 228. For example, the selection criteria 230 can indicate a threshold percentage of a given amino acid present at a respective position 206, 208, 210, 212 of the variant protein sequences 204. Additionally, the selection criteria 230 can indicate that conserved positions 228 include positions having an amino acid present in the target protein sequence 202 that is present in the variant protein sequences 204 with at least a threshold frequency. To illustrate, and continuing with the example from above for position one, a taurine can be present at position one 206 of the target protein sequence 202 and the selection criteria 230 can indicate a threshold percentage of 80%. In this situation, because taurine is present in the target protein sequence 202 at position one and because taurine is present at position one 206 of the variant protein sequences 204 at greater than an 80% frequency, position one can be included in the conserved positions 228.

The process 200 can also include determining one or more identifier sequences 232 based on the conserved positions 228. The one or more identifier sequences 232 can include a non-contiguous arrangement of amino acids at positions of the target protein sequence 202 that are included in the conserved positions 228. The one or more identifier sequences 232 can be comprised of one or more single positions of the target protein sequence 202, one or more groups of positions of the target protein sequence 202, or both one or more single positions and one or more groups of positions of the target protein sequence 202. The one or more identifier sequences 232 can then be used to identify test protein sequences that correspond to the target protein sequence 202.

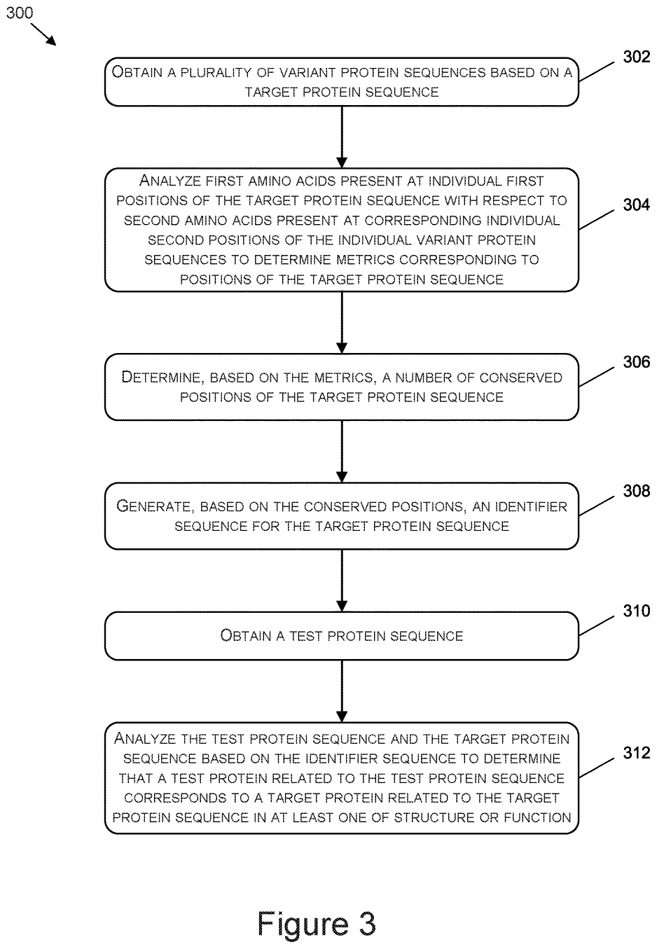

FIG. 3 is a flow diagram of an example process 300 to analyze variant protein sequences in relation to target protein sequences to determine non-contiguous sequences of the target protein that are present in the variant protein sequences, in accordance with one or more illustrative implementations.

At operation 302, the process 300 can include obtaining a plurality of variant protein sequences based on a target protein sequence. Individual variant protein sequences of the plurality of protein sequences can have a first number of conserved positions that include a same amino acid as the target protein sequence and a second number of modified positions that include a different amino acid from the target protein. In one or more examples, the plurality of variant protein sequences can be generated by executing one or more machine learning models based on the target protein sequence. In one or more examples, the one or more machine learning models can include at least one of a transformer-based machine learning model, a variational autoencoder-based machine learning model, a graph-based neural network, a generative adversarial network machine learning model, a diffusion machine learning model, or a reinforcement learning machine learning model.

In at least some examples, the variant protein sequences can be generated by determining a subset of a collection of variant protein sequences. In various examples, the collection of variant protein sequences can be generated by executing one or more machine learning techniques. In one or more examples, the subset of the collection of variant protein sequences can be determined by executing one or more computational models to determine at least one of secondary structures, three-dimensional structures, folding configurations, biophysical properties, or function of proteins corresponding to the collection of variant protein sequences. Additionally, one or more quantitative measures can be determined indicating a measure of correspondence between at least one of secondary structures, three-dimensional structures, folding configurations, biophysical properties, or function of the target protein with respect to the proteins corresponding to the collection of variant protein sequences. In one or more examples, the variant protein sequences can be determined by determining a subset of the collection of variant protein sequences based on the quantitative measures for the subset of the generated sequences satisfying one or more threshold criteria. In one or more illustrative examples, the one or more computational models executed to determine the characteristics of the variant proteins can comprise one or more machine learning models that are different from the one or more machine learning models executed to generate the collection of variant protein sequences.

In still other examples, the subset of the collection of variant protein sequences can be determined by synthesizing variant proteins that correspond to the collection of variant protein sequences and performing at least one of one or more assays or one or more analytical tests to determine characteristics of the synthesized variant proteins. In addition, an analysis can be performed of the characteristics of the synthesized variant proteins with respect to additional characteristics of a target protein corresponding to the target protein sequence. Based on the analysis, a subset of the collection of the variant protein sequences can be determined that comprises the variant protein sequences. In one or more illustrative examples, the one or more assays can indicate an amount of binding of the variant proteins to a ligand that binds to the target protein. Further, the characteristics of the variant proteins can include at least one of secondary structures, three-dimensional structures, folding configurations, biophysical properties, or function.

The process 300 can include, at operation 304, analyzing first amino acids present at individual first positions of the target protein sequence with respect to second amino acids at corresponding individual second positions of the individual variant protein sequences to determine metrics corresponding to the positions of the target protein sequence. In various examples, the metrics corresponding to positions of the target protein sequence can be determined by determining a frequency of occurrence of a plurality of individual types of amino acids at individual positions of the individual variant protein sequences.

In addition, at operation 306, the process 300 can include determining, based on the metrics, a number of conserved positions of the target protein sequence. Individual conserved positions of the number of conserved positions can have a same nucleic acid present in the target sequence as at least a threshold amount of the variant sequences.

Further, the process 300 can include, at operation 308, generating, based on the conserved positions, an identifier sequence for the target protein sequence, the identifier sequence including at least one non-contiguous region of the target protein sequence.

The process 300 can also include, at operation 310, obtaining a test protein sequence. In one or more examples, the test protein sequence can be obtained by obtaining a sample that includes one or more proteins and performing one or more sequencing operations with respect to the sample to determine the test protein sequence. The test protein sequence can correspond to a protein of the one or more proteins included in the sample.

Additionally, the process 300 can include, at operation 312, analyzing the test protein sequence and the target protein sequence to determine that a test protein related to the test protein sequence corresponds to a target protein related to the target protein sequence in at least one of structure or function.

FIG. 3 illustrates an example process for analyzing protein sequences. The example process is illustrated as collections of blocks in logical flow graphs, which represent sequences of at least some operations that can be implemented in hardware, software, or a combination thereof. The blocks are referenced by numbers. In the context of software, the blocks represent computer-executable instructions stored on one or more computer-readable media that, when executed by one or more processing units (such as hardware microprocessors), perform the recited operations. Generally, computer-executable instructions include routines, programs, objects, components, data structures, and the like that perform particular functions or implement particular data types. The order in which the operations are described is not intended to be construed as a limitation, and any number of the described blocks can be combined in any order and/or in parallel to implement the process.

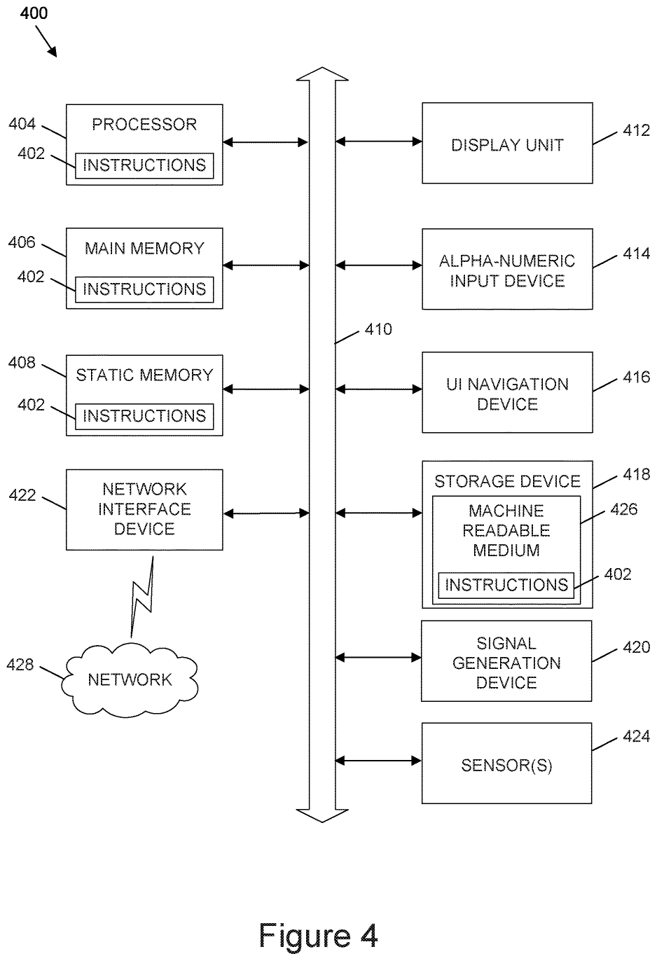

FIG. 4 illustrates a diagrammatic representation of a computing device 400 in the form of a computer system within which a set of instructions may be executed for causing the computing device 400 to perform any one or more of the methodologies discussed herein, according to an example, according to an example implementation. Specifically, FIG. 4 shows a diagrammatic representation of the computing device 400 in the example form of a computer system, within which instructions 402 (e.g., software, a program, an application, an applet, an app, or other executable code) for causing the computing device 400 to perform any one or more of the methodologies discussed herein may be executed. For example, the instructions 402 may cause the computing device 400 to implement the framework 100, described with respect to FIG. 1, to execute the operations performed by the system 102 described with respect to FIG. 1, and to execute the methods 200 and 300 described with respect to FIGS. 2 and 3, respectively.

The instructions 402 transform the general, non-programmed computing device 400 into a particular computing device 400 programmed to carry out the described and illustrated functions in the manner described. In alternative implementations, the computing device 400 operates as a standalone device or may be coupled (e.g., networked) to other machines. In a networked deployment, the computing device 400 may operate in the capacity of a server machine or a client machine in a server-client network environment, or as a peer machine in a peer-to-peer (or distributed) network environment. The computing device 400 may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 402, sequentially or otherwise, that specify actions to be taken by the computing device 400. Further, while only a single computing device 400 is illustrated, the term “machine” shall also be taken to include a collection of computing devices 400 that individually or jointly execute the instructions 402 to perform any one or more of the methodologies discussed herein.

Examples of computing device 400 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In implementations in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor implemented. For example, at least some of the operations of a method can be performed by one or more processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service”

(SaaS). For example, at least some of the operations can be performed by a group of computers (as examples of machines including processors), with these operations being accessible via a network (e.g., the Internet) and via one or more appropriate interfaces (e.g., Application Program Interfaces (APIs)).

Example implementations (e.g., apparatus, systems, or methods) can be implemented in digital electronic circuitry, in computer hardware, in firmware, in software, or in any combination thereof. Example implementations can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In implementations deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., computing device 400) and software architectures that can be deployed in example implementations.

In an example, the computing device 400 can operate as a standalone device or the computing device 400 can be connected (e.g., networked) to other machines.

In a networked deployment, the computing device 400 can operate in the capacity of either a server or a client machine in server-client network environments. In an example, computing device 400 can act as a peer machine in peer-to-peer (or other distributed) network environments. The computing device 400 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the computing device 400. Further, while only a single computing device 400 is illustrated, the term “computing device” shall also be taken to include any collection of machines that individually or jointly execute a set (or multiple sets) of instructions to perform any one or more of the methodologies discussed herein.

Example computing device 400 can include a processor 404 (e.g., a central processing unit (CPU), a graphics processing unit (GPU), or both), a main memory 406 and a static memory 408, some or all of which can communicate with each other via a bus 410. The computing device 400 can further include a display unit 412, an alphanumeric input device 414 (e.g., a keyboard), and a user interface (UI) navigation device 416 (e.g., a mouse). In an example, the display unit 412, input device 414, and UI navigation device 416 can be a touch screen display. The computing device 400 can additionally include a storage device (e.g., drive unit) 418, a signal generation device 420 (e.g., a speaker), a network interface device 422, and one or more sensors 424, such as a global positioning system (GPS) sensor, compass, accelerometer, or another sensor.

The storage device 418 can include a machine readable medium 426 on which is stored one or more sets of data structures or instructions 402 (e.g., software) embodying or utilized by any one or more of the methodologies or functions described herein. The instructions 402 can also reside, completely or at least partially, within the main memory 406, within static memory 408, or within the processor 404 during execution thereof by the computing device 400. In an example, one or any combination of the processor 404, the main memory 406, the static memory 408, or the storage device 418 can constitute machine readable media.

While the machine readable medium 426 is illustrated as a single medium, the term “machine readable medium” can include a single medium or multiple media (e.g., a centralized or distributed database, and/or associated caches and servers) that are configured to store the one or more instructions 402. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine-readable media can include non-volatile memory, including, by way of example, semiconductor memory devices (e.g., Electrically Programmable Read-Only Memory

(EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 402 can further be transmitted or received over a communications network 428 using a transmission medium via the network interface device 422 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

As used herein, a component can refer to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various example implementations, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein.

In view of the above-described implementations of subject matter this application discloses the following list of example implementations, wherein one feature of an example implementation in isolation or more than one feature of an example, taken in combination and, optionally, in combination with one or more features of one or more further examples are further examples also falling within the disclosure of this application.

Example 1. A method comprising: obtaining, by one or more computing devices each having one or more processors and memory, a plurality of variant protein sequences based on a target protein sequence, individual variant protein sequences of the plurality of variant protein sequences having a first number of conserved positions that include a same amino acid as the target protein sequence and a second number of modified positions that include a different amino acid from the target protein sequence; analyzing, by the one or more computing devices, first amino acids present at individual first positions of the target protein sequence with respect to second amino acids at corresponding individual second positions of the individual variant protein sequences to determine metrics corresponding to the individual first positions of the target protein sequence; determining, by the one or more computing devices and based on the metrics, a number of conserved positions of the target protein sequence, individual conserved positions of the number of conserved positions having a same nucleic acid present in the target protein sequence as at least a threshold amount of the plurality of variant protein sequences; generating, by the one or more computing devices and based on the individual conserved positions, an identifier sequence for the target protein sequence, the identifier sequence including at least one non-contiguous region of the target protein sequence; obtaining, by the one or more computing devices, a test protein sequence; and analyzing, by the one or more computing devices, the test protein sequence and the target protein sequence based on the identifier sequence to determine that a test protein related to the test protein sequence corresponds to a target protein related to the target protein sequence in at least one of structure or function.

Example 2. The method of example 1, comprising: obtaining, by the one or more computing devices, the target protein sequence; and executing, by the one or more computing devices, one or more machine learning models to generate the plurality of variant protein sequences based on the target protein sequence.

Example 3. The method of example 2, wherein the one or more machine learning models include at least one of a transformer-based machine learning model, a variational autoencoder-based machine learning model, a graph-based neural network, a generative adversarial network machine learning model, a diffusion machine learning model, or a reinforcement learning machine learning model.

Example 4. The method of any one of examples 1-3, comprising: obtaining, by the one or more computing devices, a collection of variant protein sequences; executing, by the one or more computing devices, one or more computational models to determine at least one of secondary structures, three-dimensional structures, folding configurations, biophysical properties, or function of proteins corresponding to the collection of variant protein sequences; and determining, by the one or more computing devices, one or more quantitative measures indicating a measure of correspondence between at least one of secondary structures, three-dimensional structures, folding configurations, biophysical properties, or function of the target protein with respect to the proteins corresponding to the collection of variant protein sequences.

Example 5. The method of example 4, comprising: determining, by the one or more computing devices, the plurality of variant protein sequences by determining a subset of the collection of variant protein sequences based on the one or more quantitative measures for the subset of the collection of variant protein sequences satisfying one or more threshold criteria.

Example 6. The method of example 4, wherein the one or more computational models include one or more machine learning models that are executable to predict at least one of secondary structures, three-dimensional structures, folding configurations, biophysical properties, or function based on protein sequences.

Example 7. The method of any one of examples 1-6, comprising: obtaining, by the one or more computing devices, a collection of variant protein sequences; synthesizing variant proteins that correspond to the collection of variant protein sequences; performing at least one of one or more assays or one or more analytical tests to determine characteristics of the variant proteins; performing, by the one or more computing devices, an analysis of the characteristics of the variant proteins with respect to additional characteristics of a target protein corresponding to the target protein sequence; and determining, by the one or more computing devices and based on the analysis, a subset of the collection of variant protein sequences that comprises the plurality of variant protein sequences.

Example 8. The method of example 7, wherein the one or more assays indicate an amount of binding of the variant proteins to a ligand that binds to the target protein.

Example 9. The method of example 7, wherein the characteristics of the variant proteins include at least one of secondary structures, three-dimensional structures, folding configurations, biophysical properties, or function.

Example 10. The method of any one of examples 1-9, comprising: obtaining a sample that includes one or more proteins; and performing one or more sequencing operations with respect to the sample to determine the test protein sequence, the test protein sequence corresponding to a protein of the one or more proteins included in the sample.

Example 11. The method of any one of examples 1-10, wherein the metrics corresponding to positions of the target protein sequence are determined by determining a frequency of occurrence of a plurality of individual types of amino acids at individual positions of the individual variant protein sequences.

Example 12. A system comprising: one or more processors; and memory storing computer-readable instructions that, when executed by the one or more processors, cause the one or more processors to perform operations comprising: obtaining a plurality of variant protein sequences based on a target protein sequence, individual variant protein sequences of the plurality of variant protein sequences having a first number of conserved positions that include a same amino acid as the target protein sequence and a second number of modified positions that include a different amino acid from the target protein sequence; analyzing first amino acids present at individual first positions of the target protein sequence with respect to second amino acids at corresponding individual second positions of the individual variant protein sequences to determine metrics corresponding to the individual first positions of the target protein sequence; determining, based on the metrics, a number of conserved positions of the target protein sequence, individual conserved positions of the number of conserved positions having a same nucleic acid present in the target protein sequence as at least a threshold amount of the plurality of variant protein sequences; generating, based on the individual conserved positions, an identifier sequence for the target protein sequence, the identifier sequence including at least one non-contiguous region of the target protein sequence; obtaining a test protein sequence; and analyzing the test protein sequence and the target protein sequence based on the identifier sequence to determine that a test protein related to the test protein sequence corresponds to a target protein related to the target protein sequence in at least one of structure or function.

Example 13. The system of example 12, wherein the memory stores additional computer-readable instructions that, when executed by the one or more processors, cause the one or more processors to perform operations comprising: obtaining the target protein sequence; and executing one or more machine learning models to generate a collection of variant protein sequences based on the target protein sequence.

Example 14. The system of example 13, wherein the memory stores additional computer-readable instructions that, when executed by the one or more processors, cause the one or more processors to perform operations comprising: executing one or more additional machine learning models to determine at least one of secondary structures, three-dimensional structures, folding configurations, biophysical properties, or function of proteins corresponding to the collection of variant protein sequences; and determining one or more quantitative measures indicating a measure of correspondence between at least one of secondary structures, three-dimensional structures, folding configurations, biophysical properties, or function of the target protein with respect to the proteins corresponding to the collection of variant protein sequences.

Example 15. The system of example 14, wherein the memory stores additional computer-readable instructions that, when executed by the one or more processors, cause the one or more processors to perform operations comprising: determining the plurality of variant protein sequences by determining a subset of the collection of variant protein sequences based on the one or more quantitative measures for the subset of the collection of variant protein sequences satisfying one or more threshold criteria.

Example 16. The system of any one of examples 13-15, wherein the one or more machine learning models include at least one of a transformer-based machine learning model, a variational autoencoder-based machine learning model, a graph-based neural network, a generative adversarial network machine learning model, a diffusion machine learning model, or a reinforcement learning machine learning model.

Example 17. The system of any one of examples 13-16, wherein the memory stores additional computer-readable instructions that, when executed by the one or more processors, cause the one or more processors to perform operations comprising: obtaining target protein data indicating characteristics of a target protein that corresponds to the target protein sequence; obtaining variant protein data indicating additional characteristics of variant proteins that correspond to at least a portion of the collection of variant protein sequences; and analyzing the characteristics of the target protein with respect to the additional characteristics of the variant proteins to determine a subset of the collection of variant protein sequences that comprises the plurality of variant protein sequences.

Example 18. The system of example 17, wherein the characteristics of the target protein and the additional characteristics of the variant proteins include at least one of secondary structures, three-dimensional structures, folding configurations, biophysical properties, or function.

Example 19. One or more non-transitory computer-readable storage media including computer-readable instructions that, when executed by one or more hardware processors, cause the one or more hardware processors to perform operations comprising: obtaining a plurality of variant protein sequences based on a target protein sequence, individual variant protein sequences of the plurality of variant protein sequences having a first number of conserved positions that include a same amino acid as the target protein sequence and a second number of modified positions that include a different amino acid from the target protein sequence; analyzing first amino acids present at individual first positions of the target protein sequence with respect to second amino acids at corresponding individual second positions of the individual variant protein sequences to determine metrics corresponding to the individual first positions of the target protein sequence; determining, based on the metrics, a number of conserved positions of the target protein sequence, individual conserved positions of the number of conserved positions having a same nucleic acid present in the target protein sequence as at least a threshold amount of the plurality of variant protein sequences; generating, based on the individual conserved positions, an identifier sequence for the target protein sequence, the identifier sequence including at least one non-contiguous region of the target protein sequence; obtaining a test protein sequence; and analyzing the test protein sequence and the target protein sequence based on the identifier sequence to determine that a test protein related to the test protein sequence corresponds to a target protein related to the target protein sequence in at least one of structure or function.

Example 20. The one or more non-transitory computer-readable storage media of example 19, including additional computer-readable instructions that, when executed by one or more hardware processors, cause the one or more hardware processors to perform additional operations comprising: determining the metrics corresponding to the individual first positions of the target protein sequence by determining a frequency of occurrence of a plurality of individual types of amino acids at individual positions of the individual variant protein sequences.

### Examples of the Disclosure

The methods and systems described herein provide increased success with respect to finding re-engineered toxins compared to, for example, detection of k-mers identified by FAST-NA methods. In an example case performed using k-mer signatures, a test set included re-engineered toxin sequences as follows: Set A: ~30K sequences with ~40% BLAST homology to the original toxin; Set B: ~30K sequences further engineered specifically for evasion; Set C: ~1.2K sequences designed to be even harder to find. Executing k-mer based detection against the re-engineered toxin sequences yielded the following results, which are relatively poor: Set A: 3 toxin identifications; Set B: 0 toxin identifications; Set C: 0 toxin identifications. Further, spot-checking of sequences showed that there were no BLAST matches longer than 4 amino acids (AAs). Table 1 illustrates example results of a test case including non-contiguous signatures against which the techniques described herein were applied. With reference to Table 1, the techniques described herein may effectively use non-contiguous signatures for identifying re-engineered toxins. In particular, nine non-contiguous signatures were identified and tested against set A, set B, and set C.

TABLE 1
Results of Non-Contiguous Signature Matching

| | | |
|---|---|---|
| Set A | 29949/29955 | 99.98% match |
| Set B | 29892/29952 | 99.80% match |
| Set C | 1127/1128 | 99.91% match |
| Viral Threat | 0/1791 | 0% false positives |
| Bacterial Threat | 0/748 | 0% false positives |
| Safe synbio sequences | 0/5183 | 0% false positives |

Accordingly, with respect to implementing some FAST-NA scanner approaches, the following problems are present: re-engineered toxins often cannot be identified via k-mer matching, low homology means even BLAST matches will be discarded under many SOPs, and sequences having a relatively high complexity are unable to be matched by BLAST at all. The results of Table 1 show the accuracy that can be achieved by the implementations described herein when non-contiguous identifier sequences are used to detect amino acid sequences of toxins that have been re-engineered.

The techniques described herein address some assumptions behind k-mer matching, such as, for example, an assumption that dangerous function derives from conserved structure and an assumption that a conserved structure is likely to have contiguous regions. That is, for example, for re-engineering (e.g., re-engineered sequences, re-engineered toxins), a conserved structure is not limited to having contiguous regions. For example, non-contiguous conserved structures are possible in re-engineered sequences.

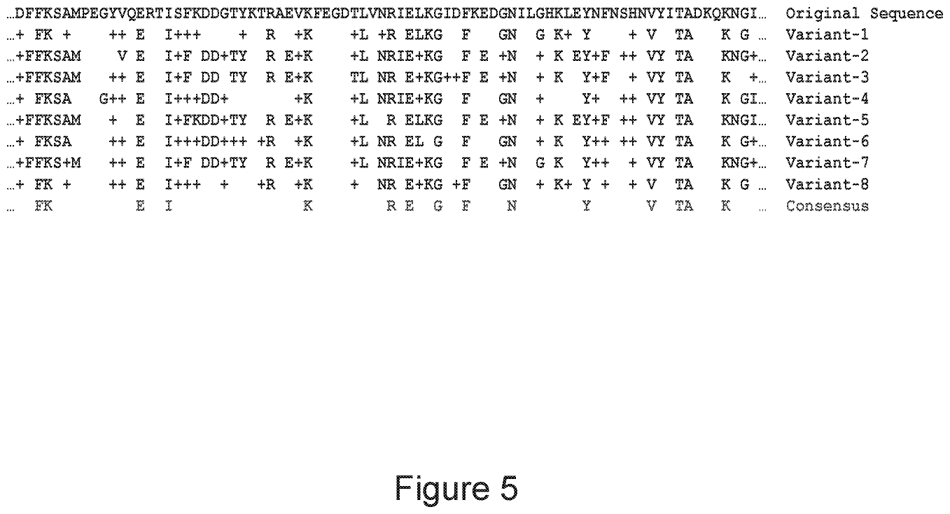

The systems and techniques described herein provide design diversification and conserved amino acid identification (e.g., as described with reference to FIG. 1, FIG. 2, and FIG. 3) supportive of generating signatures of sequences, in which gaps are present in the sequences, which thus supports effective threat detection of such sequences (e.g., re-engineered toxins, or the like). A portion of an example target protein sequence is given by DFFKSAMPEGYVQERTISFKDDGTYKTRAEVKFEGDTLVNRIELKGIDFKEDGNILGHKL EYNFNSHNVYITADKQKNGI (SEQ. ID NO: 1) in FIG. 5. Conserved positions of the target sequence shown in FIG. 5 and labeled as “Consensus” positions were identified by determining positions of variant protein sequences having the same amino acid as a corresponding position of the target sequence. FIG. 5 is a table showing conserved positions of the target sequence within a number of variant sequences. Some portions of the target sequence that are included in one or more variants include: ELKG (SEQ. ID NO: 2), FFKSAM (SEQ. ID NO: 3), NRIE (SEQ. ID NO: 4), FKSA (SEQ. ID NO: 5), FKDD (SEQ. ID NO: 6), FFKS (SEQ. ID NO: 7), and KNGI (SEQ. ID NO: 8).

The term “about” is intended to include the degree of error associated with measurement of the particular quantity based upon the equipment available at the time of filing the application.

The terminology used herein is for the purpose of describing particular implementations only and is not intended to be limiting of the present disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

While the present disclosure has been described with reference to an exemplary implementation or implementations, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the essential scope thereof. Therefore, it is intended that the present disclosure not be limited to the particular implementation disclosed as the best mode contemplated for carrying out this present disclosure, but that the present disclosure will include all implementations falling within the scope of the claims.

What is claimed is:

1. A method comprising:
obtaining, by one or more computing devices each having one or more processors and memory, a plurality of variant protein sequences based on a target protein sequence, individual variant protein sequences of the plurality of variant protein sequences having a first number of conserved positions that include a same amino acid as the target protein sequence and a second number of modified positions that include a different amino acid from the target protein sequence;
analyzing, by the one or more computing devices, first amino acids present at individual first positions of the target protein sequence with respect to second amino acids at corresponding individual second positions of the individual variant protein sequences to determine metrics corresponding to the individual first positions of the target protein sequence;
determining, by the one or more computing devices and based on the metrics, a number of conserved positions of the target protein sequence, individual conserved positions of the number of conserved positions having a same nucleic acid present in the target protein sequence as at least a threshold amount of the plurality of variant protein sequences;
generating, by the one or more computing devices and based on the individual conserved positions, an identifier sequence for the target protein sequence, the identifier sequence including at least one non-contiguous region of the target protein sequence;
obtaining, by the one or more computing devices, a test protein sequence; and
analyzing, by the one or more computing devices, the test protein sequence and the target protein sequence based on the identifier sequence to determine that a test protein related to the test protein sequence corresponds to a target protein related to the target protein sequence in at least one of structure or function.

2. The method of claim 1, comprising:
obtaining, by the one or more computing devices, the target protein sequence; and
executing, by the one or more computing devices, one or more machine learning models to generate the plurality of variant protein sequences based on the target protein sequence.

3. The method of claim 2, wherein the one or more machine learning models include at least one of a transformer-based machine learning model, a variational autoencoder-based machine learning model, a graph-based neural network, a generative adversarial network machine learning model, a diffusion machine learning model, or a reinforcement learning machine learning model.

4. The method of claim 1, comprising:
obtaining, by the one or more computing devices, a collection of variant protein sequences;
executing, by the one or more computing devices, one or more computational models to determine at least one of secondary structures, three-dimensional structures, folding configurations, biophysical properties, or function of proteins corresponding to the collection of variant protein sequences; and
determining, by the one or more computing devices, one or more quantitative measures indicating a measure of correspondence between at least one of secondary structures, three-dimensional structures, folding configurations, biophysical properties, or function of the target protein with respect to the proteins corresponding to the collection of variant protein sequences.

5. The method of claim 4, comprising:
determining, by the one or more computing devices, the plurality of variant protein sequences by determining a subset of the collection of variant protein sequences based on the one or more quantitative measures for the subset of the collection of variant protein sequences satisfying one or more threshold criteria.

6. The method of claim 4, wherein the one or more computational models include one or more machine learning models that are executable to predict at least one of secondary structures, three-dimensional structures, folding configurations, biophysical properties, or function based on protein sequences.

7. The method of claim 1, comprising:
obtaining, by the one or more computing devices, a collection of variant protein sequences;
synthesizing variant proteins that correspond to the collection of variant protein sequences;
performing at least one of one or more assays or one or more analytical tests to determine characteristics of the variant proteins;
performing, by the one or more computing devices, an analysis of the characteristics of the variant proteins with respect to additional characteristics of a target protein corresponding to the target protein sequence; and
determining, by the one or more computing devices and based on the analysis, a subset of the collection of variant protein sequences that comprises the plurality of variant protein sequences.

8. The method of claim 7, wherein the one or more assays indicate an amount of binding of the variant proteins to a ligand that binds to the target protein.

9. The method of claim 7, wherein the characteristics of the variant proteins include at least one of secondary structures, three-dimensional structures, folding configurations, biophysical properties, or function.

10. The method of claim 1, comprising:
obtaining a sample that includes one or more proteins; and
performing one or more sequencing operations with respect to the sample to determine the test protein sequence, the test protein sequence corresponding to a protein of the one or more proteins included in the sample.

11. The method of claim 1, wherein the metrics corresponding to positions of the target protein sequence are determined by determining a frequency of occurrence of a plurality of individual types of amino acids at individual positions of the individual variant protein sequences.

12. A system comprising:
one or more processors; and
memory storing computer-readable instructions that, when executed by the one or more processors, cause the one or more processors to perform operations comprising:
obtaining a plurality of variant protein sequences based on a target protein sequence, individual variant protein sequences of the plurality of variant protein sequences having a first number of conserved positions that include a same amino acid as the target protein sequence and a second number of modified positions that include a different amino acid from the target protein sequence;
analyzing first amino acids present at individual first positions of the target protein sequence with respect to second amino acids at corresponding individual second positions of the individual variant protein sequences to determine metrics corresponding to the individual first positions of the target protein sequence;
determining, based on the metrics, a number of conserved positions of the target protein sequence, individual conserved positions of the number of conserved positions having a same nucleic acid present in the target protein sequence as at least a threshold amount of the plurality of variant protein sequences;
generating, based on the individual conserved positions, an identifier sequence for the target protein sequence, the identifier sequence including at least one non-contiguous region of the target protein sequence;
obtaining a test protein sequence; and
analyzing the test protein sequence and the target protein sequence based on the identifier sequence to determine that a test protein related to the test protein sequence corresponds to a target protein related to the target protein sequence in at least one of structure or function.

13. The system of claim 12, wherein the memory stores additional computer-readable instructions that, when executed by the one or more processors, cause the one or more processors to perform operations comprising:
obtaining the target protein sequence; and
executing one or more machine learning models to generate a collection of variant protein sequences based on the target protein sequence.

14. The system of claim 13, wherein the memory stores additional computer-readable instructions that, when executed by the one or more processors, cause the one or more processors to perform operations comprising:
executing one or more additional machine learning models to determine at least one of secondary structures, three-dimensional structures, folding configurations, biophysical properties, or function of proteins corresponding to the collection of variant protein sequences; and
determining one or more quantitative measures indicating a measure of correspondence between at least one of secondary structures, three-dimensional structures, folding configurations, biophysical properties, or function of the target protein with respect to the proteins corresponding to the collection of variant protein sequences.

15. The system of claim 14, wherein the memory stores additional computer-readable instructions that, when executed by the one or more processors, cause the one or more processors to perform operations comprising:
determining the plurality of variant protein sequences by determining a subset of the collection of variant protein sequences based on the one or more quantitative measures for the subset of the collection of variant protein sequences satisfying one or more threshold criteria.

16. The system of claim 13, wherein the one or more machine learning models include at least one of a transformer-based machine learning model, a variational autoencoder-based machine learning model, a graph-based neural network, a generative adversarial network machine learning model, a diffusion machine learning model, or a reinforcement learning machine learning model.

17. The system of claim 13, wherein the memory stores additional computer-readable instructions that, when executed by the one or more processors, cause the one or more processors to perform operations comprising:
obtaining target protein data indicating characteristics of a target protein that corresponds to the target protein sequence;
obtaining variant protein data indicating additional characteristics of variant proteins that correspond to at least a portion of the collection of variant protein sequences; and
analyzing the characteristics of the target protein with respect to the additional characteristics of the variant proteins to determine a subset of the collection of variant protein sequences that comprises the plurality of variant protein sequences.

18. The system of claim 17, wherein the characteristics of the target protein and the additional characteristics of the variant proteins include at least one of secondary structures, three-dimensional structures, folding configurations, biophysical properties, or function.

19. One or more non-transitory computer-readable storage media including computer-readable instructions that, when executed by one or more hardware processors, cause the one or more hardware processors to perform operations comprising:
obtaining a plurality of variant protein sequences based on a target protein sequence, individual variant protein sequences of the plurality of variant protein sequences having a first number of conserved positions that include a same amino acid as the target protein sequence and a second number of modified positions that include a different amino acid from the target protein sequence;
analyzing first amino acids present at individual first positions of the target protein sequence with respect to second amino acids at corresponding individual second positions of the individual variant protein sequences to determine metrics corresponding to the individual first positions of the target protein sequence;
determining, based on the metrics, a number of conserved positions of the target protein sequence, individual conserved positions of the number of conserved positions having a same nucleic acid present in the target protein sequence as at least a threshold amount of the plurality of variant protein sequences;
generating, based on the individual conserved positions, an identifier sequence for the target protein sequence, the identifier sequence including at least one non-contiguous region of the target protein sequence;
obtaining a test protein sequence; and
analyzing the test protein sequence and the target protein sequence based on the identifier sequence to determine that a test protein related to the test protein sequence corresponds to a target protein related to the target protein sequence in at least one of structure or function.

20. The one or more non-transitory computer-readable storage media of claim 19, including additional computer-readable instructions that, when executed by one or more hardware processors, cause the one or more hardware processors to perform additional operations comprising:
determining the metrics corresponding to the individual first positions of the target protein sequence by determining a frequency of occurrence of a plurality of individual types of amino acids at individual positions of the individual variant protein sequences.


SEQUENCE LISTING (8 sequences)

SEQ ID NO: 1 - protein, 80 aa, synthetic construct
DFFKSAMPEGYVQERTISFKDDGTYKTRAEVKFEGDTLVNRIELKGIDFKEDGNILGHKLEYNFNSHNVYITADKQKNGI

SEQ ID NO: 2 - protein, 4 aa, synthetic construct
ELKG

SEQ ID NO: 3 - protein, 6 aa, synthetic construct
FFKSAM

SEQ ID NO: 4 - protein, 4 aa, synthetic construct
NRIE

SEQ ID NO: 5 - protein, 4 aa, synthetic construct
FKSA

SEQ ID NO: 6 - protein, 4 aa, synthetic construct
FKDD

SEQ ID NO: 7 - protein, 4 aa, synthetic construct
FFKS

SEQ ID NO: 8 - protein, 4 aa, synthetic construct
KNGI